SEQUENCE LISTING (6 sequences)

SEQ ID NO: 1 - protein, 11 aa, Oplophorus gracilirostris
  SBiT with K(d) of 190 μM
VTGYRLFEEIL

SEQ ID NO: 2 - protein, 11 aa, Oplophorus gracilirostris
  SBiT with K(d) of 0.28 μM
VTGYRLFEKIL

SEQ ID NO: 3 - DNA, 507 bp, Artificial Sequence
  DNA sequence of pG-SB
atgggctggagccatccgcagtttgaaaaaggtggtagcatgacatttaaactgattatcaacggcaaaactttaaagggagagatcacaatagaagcggtggatgcttaggaggcggagaagatttttaagcagtatgcaaatgattatggaattgatggtgaatggacttatgacgacgcaactaaaactttcacggtaacagaagaatttacaggaggttcgggtgggtcgggaggttctggcggctctggaggaagtggtggtagcggtgaattcgccgaagcagccgctaaagaagccgcagcaaaggaagccgcggccaaggaggcagccgcaaaagaggccgcggcgaaggaagcagcagccaaggcagaattcgggggtagcggcggctcggggggtagtggtggaagcgggggttcaggcggttctgggggtaccgttaccggctatcgtctgtttgaaaaagagagcggcggttcacatcatcatcaccaccattaa

SEQ ID NO: 4 - DNA, 948 bp, Artificial Sequence
  DNA sequence of pG-LB
atgggctggagccatccgcagtttgaaaaaggtggtagcatgacatttaaactgattatcaacggcaaaactttaaagggagagatcacaatagaagcggtggatgcttaggaggcggagaagatttttaagcagtatgcaaatgattatggaattgatggtgaatggacttatgacgacgcaactaaaactttcacggtaacagaagaatttacaggaggttcgggtgggtcgggaggttctggcggctctggaggaagtggtggtagcggtgaattcgccgaagcagccgctaaagaagccgcagcaaaggaagccgcggccaaggaggcagccgcaaaagaggccgcggcgaaggaagcagcagccaaggcagaattcgggggtagcggcggctcggggggtagtggtggaagcgggggttcaggcggttctgggggtaccgtcttcacactcgaagatttcgttggggactgggaacagacagccgcctacaacctggaccaagtccttgaacagggaggtgtgtccagtttgctgcagaatctcgccgtgtccgtaactccgatccaaaggattgtccggagcggtgaaaatgccctgaagatcgacatccatgtcatcatcccgtatgaaggtctgagcgccgaccaaatggcccagatcgaagaggtgtttaaggtggtgtaccctgtggatgatcatcactttaaggtgatcctgccctatggcacactggtaatcgacggggttacgccgaacatgctgaactatttcggacggccgtatgaaggcatcgccgtgttcgacggcaaaaagatcactgtaacagggaccctgtggaacggcaacaaaattatcgacgagcgcctgatcacccccgacggctccatgctgttccgagtaaccatcaacagcggaggttcccaccaccaccatcaccactaa

SEQ ID NO: 5 - DNA, 594 bp, Artificial Sequence
  DNA sequence of K9C/D157C/G191C variant
atgtggtctcatcctcaatttgaatgcatggtatttactcttgaagattttgtcggtgattggcgccagaccgccggctataacctggaccaagtgcttgaacagggcggggttagcagcctgtttcaaaacctgggggtgagtgtcacgccaattcagcgcatcgttctgtcgggagagaatggtctgaaaatcgatatccacgtcattatcccgtacgaaggtctttctggtgatcagatggggcagatagaaaaaatattcaaagtggtgtacccagtagacgatcatcacttcaaggttatactgcactatggcaccctcgttatcgatggcgttactccgaatatgatcgattactttgggcgtccttatgaaggtattgcggtgttcgacggtaaaaaaattacggttaccgggacgctctggaatggtaataaaatcattgatgagcgcttgataaacccatgtggcagccttctgttcagagttacgataaacggggttacgggttggcgactgagcgaaagaatattagctgcgtccgcactcgagctgccagaaaccggttgtcaccaccaccaccaccactga

SEQ ID NO: 6 - DNA, 1149 bp, Artificial Sequence
  DNA sequence of TNFa-SB
atgtcggactcagaagtcaatcaagaagctaagccagaggtcaagccagaagtcaagcctgagactcacatcaatttaaaggtgtccgatggatcttcagagatcttcttcaagatcaaaaagaccactcctttaagaaggctgatggaagcgttcgctaaaagacagggtaaggaaatggactccttaagattcttgtacgacggtattagaattcaagctgatcagacccctgaagatttggacatggaggataacgatattattgaggctcacagagaacagattggtggtgtgcgtagcagcagccgtaccccgagcgataaaccggtggcgcatgtggtggcgaatccgcaggcggaaggccagctgcagtggctgaaccgtcgtgcgaatgcgctgctggccaacggcgtggaactgcgtgataatcagctggttgtgccgagcgaaggcctgtatctgatttatagccaggtgctgtttaaaggccagggctgcccgagcacccatgtgctgctgacccataccattagccgtattgcggtgagctatcagaccaaagtgaacctgctgtctgcgattaaaagcccgtgccagcgtgaaaccccggaaggcgcggaagcgaaaccgtggtatgaaccgatttatctgggcggcgtgtttcagctggaaaaaggcgatcgtctgagcgcggaaattaaccgtccggattatctggattttgcggaaagcggccaggtgtattttggcattattgcgctgggatccggggggagcggtggtagcggtgggtccgggggaagtggtggcagtggtgggtccggagggagtggcgggtcgggaggaagcgggggctcaggatccgggggcagtggcggctcaggaggttctggaggctcaggcggaagcgggggtagcgaagcagccgcaaaagaagcagccgccaaggaagcagcagcaaaggaggcagctgcaaaggaggcggcggcgaaagaggcggcagccaaaggaggatctggtggctcaggtggtagtggaggatcaggaggtagtggcggatcaggtaccgttaccggctatcgtctgtttgaaaaagagagcggtggttcagggggctcgtggtcacatccgcaatttgaaaagtaa

(19) United States
(12) Patent Application Publication
(10) Pub. No.: US 2022/0348984 A1
(43) Pub. Date: Nov. 3, 2022

(54) RATIOMETRIC DETECTION OF LUCIFERASE ASSAYS USING A CALIBRATOR LUCIFERASE

(71) Applicant: TECHNISCHE UNIVERSITEIT EINDHOVEN, Eindhoven (NL)

(72) Inventors: Maarten MERKX, Eindhoven (NL); Yan NI, Eindhoven (NL); Leonardus Josephus Van IJzendoorn, Eindhoven (NL)

(21) Appl. No.: 17/621,497

(22) PCT Filed: Jun. 22, 2020
(86) PCT No.: PCT/NL2020/050406

§ 371 Date: Dec. 21, 2021

Related U.S. Application Data
(60) Provisional application No. 62/893,322, filed on Aug. 29, 2019.
(60) Provisional application No. 62/864,583, filed on Jun. 21, 2019.

(51) Int. Cl. C12Q 1/66; G01N 33/58

(52) U.S. Cl. CPC C12Q 1/66; G01N 33/582

(57) ABSTRACT

The present invention relates to a ratiometric bioluminescent assay for the quantification of an analyte of interest, comprising a detector luciferase that is reactive to a substrate to emit light at a first wavelength and wherein the detector luciferase is responsive to the analyte of interest and a calibrator luciferase which is reactive to the same substrate to emit light at a second wavelength which second wavelength is different from the first wavelength emitted by the detector luciferase. The invention further relates to a method for quantifying an analyte of interest in a sample using the bioluminescent assay of the present invention and wherein the quantification of the analyte of interest is based on the calibrated ratio of measured bioluminescence intensities of the detector luciferase and the calibrator luciferase. Further, the invention relates to the use of the bioluminescent assay of the present invention in a method for quantifying an analyte of interest in a sample.

### TECHNICAL FIELD

The present invention relates to a bioluminescent assay for the ratiometric quantification of an analyte of interest comprising a detector luciferase that is responsive to the analyte of interest. The invention further relates to a method for quantifying an analyte of interest in a sample using the bioluminescent assay of the present invention and to the use of the bioluminescent assay of the present invention in a method for quantifying an analyte of interest in a sample.

### BACKGROUND

Detection of biomarkers, such as protein biomarkers provides a useful tool for disease diagnosis and treatment monitoring. Classical immunoassays such as ELISA rely on a laborious procedure involving multiple washing and incubation steps. Alternative methodologies that empower single-step homogeneous immunoassays are more attractive and desirable for point-of-care tests that can be performed outside of the laboratory setting by non-expert users.

Split protein complementation is widely validated for studying protein-protein interactions and is an interesting alternative for developing sandwich immunoassays by placing the reporter fragments on a pair of antibodies that each bind the antigen targets. In such way, antigen binding brings the fragments in close proximity and thus allows reassembly of the active reporter, which quantitatively indicates the presence of the antigens. Split reporter enzymes, in particular split luciferases, are useful luminescent reporters that benefit from a great sensitivity. A particular useful split luciferase is split NanoLuc being a highly stable and bright, blue light producing luciferase that can be split into two fragments (so called Binary Technology also referred to as ‘BiT’), an 18 kDa large BiT (LB) and a 1.3 kDa small BiT (SB), which were designed as a binary complementation reporter to study protein interactions.

Such split reporter enzymes, in particular split luciferases, have become important reporter systems to interrogate protein-protein interactions and high throughput drug screening. More in general, bioluminescent detection using luciferases is increasingly being used for in vivo imaging and point of care diagnostics.

Unlike fluorescence detection, bioluminescence does not require external excitation, making it highly suitable for sensitive detection in complex media. A drawback of bioluminescent detection is that signal intensity is determined by many factors, including the concentration of both the luciferase and the substrate, product and environmental conditions including temperature, pH, and ionic strength. Since the substrate is turned over in time, the signal is time dependent, making quantitative measurements challenging.

Quantitative measurements based on bioluminescence activity require measurement of a calibration curve under conditions that closely mimic the conditions of the assay, including the amount of active enzyme, buffer conditions, substrate concentration, and temperature and incubation time. However, in many applications these parameters are not exactly known or cannot be easily controlled. In particular the time dependence of the luminescent signal is difficult to control, especially at higher luciferase activities. One remedy has been to choose assay conditions such that the luciferase activity is kept low, either by decreasing the enzyme concentration or by using caged substrates which result in a slow release of active substrate over time. However, these solutions are far from ideal, as the former limits the range of luciferase concentrations and signal intensity, while the latter depends on yet another time-dependent process. For this reason, bioluminescent assays have so far been semi-quantitative at best, making them popular for screening-based approaches but more challenging for accurate analytical applications.

### SUMMARY OF THE INVENTION

The invention provides a method to allow internal calibration of bioluminescent assays based on (split) luciferases, which allows these assays to be used for quantitative measurements. The invention provides a solution by calibrating the bioluminescence intensity of a first luciferase (also referred to herein as a ‘reporter luciferase’ or a ‘detector luciferase’) to that of a second luciferase (also referred to herein as a ‘calibrator luciferase’) that is added to the same sample, uses the same luciferase domain and substrate, but emits light at a different wavelength.

The idea is to include in the assay a variant, i.e. the calibrator luciferase, of the luciferase used in the assay that is functionalized with a fluorescent acceptor. This calibrator luciferase contains the same luciferase domain as is used in the assay, but its energy is transferred to the fluorescent acceptor, resulting in emission at a different wavelength as the detector luciferase used in the assay. Since the calibrator luciferase does not respond to the target analyte, its activity (as determined by measuring the emission at the wavelength of the fluorescent acceptor) can be used to normalize the emission of the detector luciferase. This invention provides a generic solution to make bioluminescent assays and imaging more quantitative using ratiometric detection of the detector luciferase emission at one wavelength relative to the calibrator luciferase emission detected at a different wavelength. The assumption behind this approach is that variations in substrate concentration, product inhibition, matrix composition and temperature will affect the detector luciferase and the calibrator luciferase to the same extend.

### DESCRIPTION

The present invention provides a bioluminescent assay for the ratiometric quantification of an analyte of interest. In order to provide a time and concentration independent quantification of an analyte of interest, the bioluminescent assay of the present invention comprises a detector luciferase that is reactive to a substrate to emit light at a first wavelength and wherein the detector luciferase is responsive to the analyte of interest and a calibrator luciferase. The calibrator luciferase of the present invention is reactive to the same substrate to emit light at a second wavelength which second wavelength is different from the first wavelength emitted by the detector luciferase. In a variant of the present invention, the calibrator luciferase is a variant of the detector luciferase and contains the same luciferase domain as the detector luciferase.

By providing a bioluminescent assay comprising a detector luciferase that is responsive to the analyte of interest and a calibrator luciferase that is reactive to the same substrate as the detector luciferase, the calibrator luciferase is able to catalyse the oxidation of the same substrate as is catalysed by the detector luciferase. For example, in case the detector luciferase is a NanoLuc luciferase that catalyses the oxidation of the substrate furimazine (to generate blue light), the corresponding luciferase domain of the calibrator luciferase, e.g. a NanoLuc-mNeonGreen fusion protein, catalyses oxidation of the same substrate furimazine (to generate green light). By using a detector luciferase and a calibrator luciferase having the same luciferase domain, deviations in, for example, substrate concentrations are automatically corrected for by calibrating the ratio of measured bioluminescence intensity of the detector luciferase and measured bioluminescence intensity of the calibrator luciferase. By using a detector luciferase according to the present invention the emission ratio of measured bioluminescence intensity of the detector luciferase and measured bioluminescence intensity of the calibrator luciferase remains stable over time and can therefore be reliably detected as long as sufficient light is emitted by both the detector luciferase and calibrator luciferase.

In order to emit light at a different wavelength than the detector luciferase the calibrator luciferase may be functionalized with a fluorescent acceptor to allow energy transfer from the luciferase domain of the calibrator luciferase to the fluorescent acceptor. Such a fluorescent acceptor may be selected from the group consisting of an mNeonGreen protein, a fluorescent protein, a Cy3, a fluorescent dye, a fluorescent quantum dot, a fluorescent nanoparticle, or a carbon dot. For example, in case a NanoLuc luciferase detector and calibrator luciferase is selected, in order to provide a blue/green shifted calibrator luciferase, the preferred fluorescent acceptor is an mNeonGreen protein. Alternatively, in order to provide a blue/red shifted NanoLuc calibrator luciferase, the preferred fluorescent acceptor is a Cy3.

In order to avoid interference with the detector activity of the detector luciferase, the calibrator luciferase is non-responsive to the analyte of interest. Preferably, in order to provide an optimal system the calibrator luciferase is responsive to the same components comprised in a sample to be analysed as the detector luciferase is responsive to, except for the responsiveness to the analyte of interest. By providing such calibrator luciferase, any response to other analytes or components comprised in the sample by the detector luciferase is automatically corrected for by calibrating the ratio of measured bioluminescence intensity of the detector luciferase and measured bioluminescence intensity of the calibrator luciferase.

Although the use of a NanoLuc luciferase domain is preferred due to its bright blue emission characteristics, other luciferase domains may be suitable as well. For example, the luciferase domain of the detector luciferase and calibrator luciferase may be selected from the group consisting of luciferase domains of NanoLuc luciferase, Firefly luciferase, Renilla luciferase, Gaussia luciferase, TurboLuc luciferase and Aluc luciferase.

The analyte of interest of the sample to be analysed, may be any analyte of interest, and may be relevant for diagnosing or treatment monitoring. However, other applications, such as the detection of food toxins, water quality, and the like, may also be possible. Preferred analytes of interest may be selected from the group consisting of an antigen, an antibody, and a ligand.

Any type of bioluminescent assay may be used in order to provide the time-independent and concentration-independent quantification assay of the present invention. As long as a calibrator luciferase is selected having the same luciferase domain as the detector luciferase, detection and quantification of the analyte of interest of a sample to be analysed can be performed in a reliable manner. For example, the bioluminescent assay may be a bioluminescent sandwich immunoassay, or other types of immunoassays such as competitive immunoassays, sensor proteins based on allosteric modulation of luciferase activity or reversible control a luciferase inhibition, transcription regulation assays, assays screening for protein-protein interactions, DNA or RNA detection assays.

In an aspect of the present invention, the invention relates to the use of the bioluminescent assay according to the present invention in a method for quantifying an analyte of interest in a sample.

In a further aspect of the present invention, the invention relates to the use of a calibrator luciferase in the bioluminescent assay according to the present invention in a method for quantifying an analyte of interest in a sample.

In another aspect of the present invention, the invention relates to a method for quantifying an analyte of interest in a sample. The method comprises the steps of:

a) providing a sample comprising the analyte of interest;

b) providing a bioluminescent assay according to the invention;

c) measuring the bioluminescence intensity of the detector luciferase and the bioluminescence intensity of the calibrator luciferase for the sample provided in step a);

d) calibrating the ratio of the measured bioluminescence intensity of the detector luciferase and the bioluminescence intensity of the calibrator luciferase; and

e) quantifying the analyte of interest based on the ratio calibrated in step d).

As already explained above, it was found that the above-mentioned method provides a robust, reliable, time-independent and concentration-independent method for quantifying the analyte of interest in a sample.

The present invention further provides a method for quantifying an analyte of interest in a sample, comprising the steps of:

i) providing a sample comprising the analyte of interest;

ii) providing a detector luciferase and a calibrator luciferase, wherein:
the detector luciferase is reactive to a substrate to emit light at a first wavelength and wherein the detector luciferase is responsive to the analyte of interest;
the calibrator luciferase is reactive to the same substrate to emit light at a second wavelength which second wavelength is different from the first wavelength emitted by than the detector luciferase,

iii) measuring the bioluminescence intensity of the detector luciferase and the bioluminescence intensity of the calibrator luciferase for the sample provided in step i);

iv) calibrating the ratio of the measured bioluminescence intensity of the detector luciferase and the bioluminescence intensity of the calibrator luciferase; and

v) quantifying the analyte of interest based on the ratio calibrated in step iv).

In a preferred embodiment of the present invention, the calibrator luciferase as used in the method of the present invention is a variant of the detector luciferase and contains the same luciferase domain as the detector luciferase.

### EMBODIMENTS

In one embodiment of the present invention the present invention relates to a bioluminescent sandwich immune assay that can be performed directly in solution without any washing or incubation steps. In such assay, two antibodies that recognize different epitopes on the analyte of interest are conjugated to the large or small portions of split Nanoluc luciferase by photo-cross-linking of a protein G domain fused to the split NanoLuc part via a flexible peptide linker. In the presence of the analyte of interest a ternary complex is formed of the analyte with the two antibodies, allowing complementation of the two split luciferase parts and reconstitution of luciferase activity. This system by itself generates an intensiometric signal where the intensity of the blue light emitted by the reconstituted luciferase is a measure of the amount of analyte. However, the intensity of the blue luminescence is not constant and decreases in time, in particular for the high analyte concentrations. The problem was resolved by spiking in a low concentration of a NanoLuc-mNeonGreen fusion protein, which does not emit in the blue (460 nm) but emits green light (515 nm). The ratio of green and blue light was found to be stable in time, allowing the emission ratio to be used as a reliable measure of the analyte concentration. Ratiometric detection in combination with split NanoLuc complementation provides a very attractive and fast alternative to the currently used heterogeneous sandwich immunoassay. The method may be used for the detection of cardiac troponin I (a marker for heart attack), C-reactive protein and anti-cetuximab antibodies, however, other applications of the method may be suitable as well including the detection of other antibodies and protein biomarkers.

Other interesting applications of split reporter luciferases is in high throughput detection of protein-protein interactions. In general, the method provides a convenient and much more robust method to calibrate any bioluminescent assay.

Embodiments of the invention can be applied as a sensor strategy to develop assays and point-of-care diagnostics for several other interesting biomarkers and combine the assay with paper- or thread based diagnostic devices, for which ratiometric detection is of key importance. The method itself can be further varied by exploring calibrator variants with a more red-shifted emission signal (better separation from signal of detector luciferase). The invention can also be applied more broadly beyond in vitro assays (with luciferases), as it provides a general principle to calibrate an intensiometric bioluminescent signal. Applications envisioned include the use in cell-based high throughput screening, transcriptional reporter assays, (in vivo) bioluminescent imaging applications and novel ways to develop ratiometric bioluminescent sensor proteins.

Although the examples provided herein involve a split luciferase, the detector luciferase could be any luciferase whose activity or concentration is regulated by of the presence of an analyte. With that, the invention is also applicable to some non-split luciferases.

The present invention relates to an alternative ratiometric bioluminescent sandwich immunoassay format based on complementation of a split reporter luciferase, where the two fragments of the luciferase are conjugated to a pair of antibodies that each recognize a different epitope on the target molecule, allowing for a homogeneous sandwich immunoassay in solution (see also: Ni, Y. and M. Merkx, Ratiometric detection of luciferase assays using a calibrator luciferase. provisional patent application 62/864583, manuscript in preparation, 2020). A protein G-mediated photo-conjugation strategy (see also: Hui, J. Z., et al., LASIC: Light Activated Site-Specific Conjugation of Native IgGs. Bioconjugate Chem. 2015, 26, 8, p. 1456-1460) was introduced to allow site-specific covalent bond formation between native antibodies and split NanoLuc fragments (see also: Dixon, A. S., et al., NanoLuc Complementation Reporter Optimized for Accurate Measurement of Protein Interactions in Cells. ACS Chemical Biology, 2016. 11(2): p. 400-408), providing a generic and straightforward method applicable to a wide range of commercially available monoclonal antibodies (FIG. 1A). Moreover, the system was made ratiometric by introducing NanoLuc-mNeonGreen (see also: Suzuki, K., et al., Five colour variants of bright luminescent protein for real-time multicolour bioimaging. Nature Communications, 2016. 7: p. 1-10) as a calibrator luciferase to allow for time and concentration independent measurements. While this system could also be used for antibody detection, in that case the assay showed a relatively small increase in luciferase complementation and a bell-shaped antibody response curve (also known as the hook effect). These properties are inherent to a solution sandwich immunoassay targeting two antigen binding sites within the same detection antibody, and restrict the analytical performance of the assay (see also: Ni, Y. and M. Merkx, Ratiometric detection of luciferase assays using a calibrator luciferase. provisional patent application 62/864583, manuscript in preparation, 2020). The alternative assay format successfully addresses these shortcomings. Key to this new assay format is that one part of the split luciferase is genetically fused to the target antigen, whereas the complementary part is fused to a monoclonal antibody that specifically recognizes the antibody or the antibody-antigen complex. Proof of principle for this assay format is provided by developing a ratiometric bioluminescent assay for the detection of adalimumab.

In a further embodiment the invention relates to ratiometric bioluminescent immunoassays for TNFα binding antibodies. Antibodies binding TNFα, a pro-inflammatory cytokine that is overexpressed in inflammatory diseases, are an important example of therapeutic antibodies. TNFα actually forms a homotrimer in solution and therapeutic antibodies such as adalimumab bind TNFα by recognizing a complex discontinuous epitope formed by the interface of two monomers. Therapeutic antibodies that target TNFα such as infliximab and adalimumab have been highly successful in treating diseases such as such as Rheumatic Arthritis and Inflammatory Bowel Disease, and represent some of the top-selling drugs.

### EXAMPLES

### Example 1

### Bioluminescent Sandwich Immune Assay for the Detection of Cardiac Troponin I, C-Reactive Protein and Anti-Cetuximab Antibodies

### Cloning

The pET28a(+) vectors containing DNA encoding protein G-SB (pG-SB) and protein G-LB (pG-LB) were ordered from GenScript. Site directed mutagenesis to change SB sequences were carried using the QuikChange Lightning Site-Directed Mutagenesis kit (Agilent technologies) using specific primers. All cloning and mutagenesis results were confirmed by Sanger sequencing (StarSEQ). FIGS. 7, 9 and 10 show the DNA and corresponding amino acid sequences of pG-SB (variants) and pG-LB.

### Protein Expression

The pET28a plasmids encoding pG-LB or pG-SB were co-transformed into E. coli BL21 (DE3) together with a pEVOL vector encoding a tRNA/tRNA synthetase pair in order to incorporate para-benzoylphenylalanine (pBPA). The pEVOL vector was a gift from Peter Schultz (Addgene plasmid #31186). Cells were cultured in 2YT medium (16 g peptone, 5 g NaCl, 10 g yeast extract per liter) containing 30 pg/mL kanamycin and 25 pg/mL chloramphenicol. Protein expression was induced using 0.1 mM IPTG and 0.2% arabinose in the presence of 1 mM pBPA (Bachem, F-2800.0001). After overnight expression, cells were harvested and lysed using the Bugbuster reagent (Novagen). Proteins were purified using Ni-NTA affinity chromatography followed by Strep-Tactin purification according to the manufacturer's instructions. Protein purity was confirmed by SDS-PAGE. Correct incorporation of pBPA was confirmed by Q-ToF LC-MS. Purified proteins were stored at −80° C. until use.

The NanoLuc-mNeonGreen fusion protein was prepared as described previously (Suzuki, K., et al., Five colour variants of bright luminescent protein for real-time multicolour bioimaging. Nature Communications, 2016. 7: p. 1-10). Proteins were purified using Ni-NTA affinity chromatography and Strep-Tactin purification. Purified proteins were stored at −80° C. until use.

### Photo-Conjugation

Cardiac Troponin I antibodies 19C7 (4T21) and 4C2 (4T21), and C-reactive protein antibodies C6 (4C28cc) and C135 (4C28) were ordered from Hytest. Therapeutic antibody cetuximab was obtained via the Catherina hospital pharmacy in Eindhoven, the Netherlands. Photo-conjugation reactions were performed using a Promed UVL-30 UV light source (4×9 watt). Samples containing antibody and pG-LB or pG-SB in PBS buffer (pH7.4) were illuminated with 365 nm light for 30 to 180 minutes. The samples were kept on ice during photo-conjugation. The photo-conjugated products were further purified using Ni-NTA spin columns followed by PD G10 desalting columns or protein G spin columns.

### Luminescent Assays

Cardiac Troponin I (8T53) and C-reactive protein (8C72) were ordered from Hytest. Anti-cetuximab (HCA221) was ordered from Bio-rad. Intensiometric assays were performed at sensor proteins concentrations of 0.1-10 nM in a total volume of 15 μL in PerkinElmer flat white 384-well Optiplate. After incubation of sensor proteins and analytes for 0.5 hours, NanoGlo substrate (Promega, N1110) was added at a final dilution of 300- to 1000-fold. Luminescence intensity was recorded on Tecan Spark 10M plate reader with an integration time of 100 ms. For ratiometric assays, 1-10 ρM NanoLuc-mNeonGreen fusion protein was added into the samples and luminescence spectra were recorded between 398 nm and 653 nm with a step size of 15 nm, a bandwidth of 25 nm and an integration time of 1000 ms.

The luminescence signal was also recorded by using a digital camera. The plate was placed into a styrofoam box to exclude the surrounding light. The pictures were taken through a hole in the box using a SONY DSC-RX100 digital camera with exposure times of 30 s, F value of 1.8 and ISO value of 1600-3200. The images were analyzed by using ImageJ to calculate the absolute intensity and ratio values between the average intensity in the blue and green color channel.

### Example 1a

### Sensor Design and Characterization (Cardiac Troponin I)

In order to establish a proof-of-concept, cardiac troponin I (cTnO) as a target antigen was chosen which is an important marker for cardiac damage and requires highly sensitive detection at μM concentrations. An LB fragment of split NanoLuc and an SB fragment with a K_{d} of 2.5 μM, respectively, were fused to protein G via a semi-flexible linker. A His-tag at N-terminus and a strep-tag at C-terminus were included to facilitate the purification of the fusion proteins. The plasmid contained a TAG amber stop codon at position 24 in protein G, and co-expression with the orthogonal tRNA synthetase/tRNA pair allowed the incorporation of the unnatural amino acid para-benzyol-phenylalanine (BPA), a photo-reactive group, at the desired position (see also: Hui, J. Z., et al., LASIC: Light Activated Site-Specific Conjugation of Native IgGs. Bioconjugate Chem. 2015, 26, 8, p. 1456-1460). The BPA incorporated protein G domain can be crosslinked to the Fc domain of an antibody with 365 nm light illumination. The purified pG-LB and pG-SB proteins were photo-conjugated to a pair of anti-cTnI antibodies,19C7 and 4C2, that are compatible in sandwich immunoassays (FIG. 11A). One feature of protein G-based conjugation is that both IgG heavy chains can be modified, so a mixture of mono-conjugated, di-conjugated and non-conjugated antibodies as well as pG-LB/SB was obtained. The conjugation efficiency was affected by using various molar ratios of adapter protein to antibody (FIG. 12). Since the presence of pG-LB and pG-SB contributes to the background signal, we used an equal molar ratio of protein to antibody to minimize the unconjugated pG-LB and pG-SB in the mixture. A step of Ni affinity chromatography was used to remove the unconjugated antibody (FIG. 11B).

When 1 nM of the purified antibody-conjugated sensor proteins were incubated with cTnI in the pico- to nanomolar regime, the recorded luminescence intensity followed a bell-shaped, cTnI-dependent curve with hook effect at very high concentrations of cTnI (FIG. 13A). The maximum luminescence intensity observed at 10 nM of cTnI was more than 300-fold higher compared to the blank. The limit of detection was determined to be 5 μM (FIG. 13B). The signal response could be modulated by varying the concentrations of the sensor proteins (FIG. 13C). The use of higher concentrations of 4C2-SB could effectively shift the “hook” to relatively higher analyte concentrations. The maximum intensity was obtained at 50 nM of cTnI by using 1 nM of 19C7-LB and 10 nM of 4C2-SB. A higher S/B ratio at low analyte concentrations was obtained by using 0.1 nM 19C7-LB and 1 nM 4C2-SB, although these intensities were too low to be detected by a digital camera. Therefore, 1 nM of each sensor protein was employed in the further experiments. The effect of the split NanoLuc interaction affinities on the signal response was further investigated. The other two SB variants with K_{d} of 190 μM and 0.28 μM were tested for this purpose. The highest signal intensity was obtained using SB with K_{d} of 2.5 μM with a highest SIB ratio (FIG. 14).

One drawback of using the absolute luminescence intensity is that the signal intensity depends on many factors including substrate concentration, pH, temperature and ionic strength and typically decreases in time as a result of substrate turn-over. Indeed, the monitoring of the absolute luminescence intensity for a period of 30 minutes revealed that the signal changed in time (FIG. 13D). This is particularly problematic for point-of-care applications where it may be difficult to carefully calibrate the assay for variations in sensor concentration, substrate turn-over and environmental conditions. A ratiometric system was further developed by adding an additional calibrator luciferase. The assay mixtures were spiked with a bioluminescent NanoLuc-mNeonGreen fusion protein which can catalyse oxidation of the same substrate furimazine and generate green light (Suzuki, K., et al., Five colour variants of bright luminescent protein for real-time multicolour bioimaging. Nature Communications, 2016. 7: p. 1-10). In such way, the emission ratio as a function of antigen concentration negates the influence of the substrate concentration and the reaction conditions. As shown in FIG. 14A, the ratiometric system displayed a similar antigen dose-dependency as that obtained by the intensiometric assays. More interestingly, the emission ratio remains stable over time (FIG. 14B) and therefore can be reliably detected as long as sufficient light is emitted. The time-independency of the ratiometric system represents an important aspect of sensor performance for practical applications.

### Example 1b

### Sensor Design and Characterization (C-Reactive Protein)

The assay format of example 2 was also applied for the detection of the inflammatory marker C-reactive protein (CRP). The pG-SB and pG-LB proteins were conjugated to a pair of anti-CRP antibodies C135 (mIgG2b) and C6 (mIgG2a), respectively. Following the successful photo-conjugation of antibodies with pG-SB and pG-LB and one-step nickel affinity purification, the intensiometric assays for CRP were carried out, yielding a maximal S/B ratio of 56 (FIG. 15A). Addition of the NL-mNG calibrator made the assays less fluctuant over time without influence on the detection limit and regime (FIG. 15C and FIG. 15D). The maximal ratio change of 18 and a LOD of 3 μM were achieved in the ratiometric assays by adding 2 μM of NL-mNG calibrator.

### Assay Evaluation

The analytical performance of the assay was compared with a commercially available ELISA for quantification of CRP in human blood plasma. The test CRP samples were prepared at low mg L⁻¹ level referring to high-sensitivity CRP (hsCRP) which is clinically used for cardiovascular risk assessment. Due to the high sensitivity of our assay, the test blood plasma samples were diluted 50 times in buffer and then measured by comparing the measured blue/green ratio with the calibration curve obtained in FIG. 15C. In parallel, the CRP concentration in the test samples were determined by ELISA after appropriate dilution. A good correlation (R²=0.9906) was observed between the ELISA and our assay (FIG. 16), pointing to the accuracy of our assay and its good potential for the analysis of clinical samples. It is worth mentioning that the simple “mix-and-measure” workflow of our assay eliminates the need for multiple wash steps and thus substantially reduces total assay times to less than 1 hour compared to ELISA.

### Example 1c

### Sensor Design and Characterization (Anti-Cetuximab)

The generic sensor format of example 1 was investigated whether it could be extended to anti-antibodies monitoring. The administration of therapeutic antibodies for cancer or inflammation therapy can induce an immune response and lead to the production of anti-antibodies inactivating the therapeutic effects of the treatment. Therefore development of a simple and fast assay to detect anti-antibodies is important for assessing immunogenicity to therapeutic antibodies. An anti-cancer therapeutic antibody cetuximab was chosen as the detecting molecule which was photo-conjugated separately with pG-LB and pG-SB. The split NanoLuc functionalized cetuximab was then utilized for the luminescent detection of anti-cetuximab and a bell-shaped dose-dependency curve was obtained with the maximum S/B ratio of 8 (FIG. 17A). Addition of NanoLuc-mNeonGreen fusion protein in the ratiometric system yielded a similar assay curve (FIG. 17C) but more stable sensor signal of emission ratio over time (FIG. 17D).

### Conclusion

A generic homogenous immunoassay format based on the complementation of split Nanoluc luciferase was developed for direct detection of protein biomarkers in solution. Proof of concept was provided by successful detection of cTnI at μM concentrations. The high modularity of the sensor format enables assay of various protein targets by placing the split NanoLuc fragments on the target-binding antibodies. Moreover, a ratiometric assay system was developed by adding a calibrator luciferase to allow for time and substrate concentration independent measurements.

### Example 2

### Ratiometric Bioluminescent Immunoassays for TNFα Binding Antibodies

### Cloning

The pET28a(+) vector containing DNA encoding protein G-LargeBit (pG-LB) and the pET28a(+) vector containing DNA encoding His-SUMO-TNFα-linker-SB were ordered from GenScript. FIG. 7 and FIG. 8 show the DNA and corresponding amino acid sequences of pG-LB and SUMO-TNFα-SB, respectively.

### Protein Expression and Purification

The pET28a plasmid encoding pG-LB was co-transformed into E. coli BL21 (DE3) together with a pEVOL vector encoding a tRNA/tRNA synthetase pair in order to incorporate para-benzoyl-phenylalanine (pBPA). The pEVOL vector was a gift from Peter Schultz (Addgene plasmid #31186). Cells were cultured in 2YT medium (16 g peptone, 5 g NaCl, 10 g yeast extract per liter) containing 30 pg/mL kanamycin and 25 μg/mL chloramphenicol. Protein expression was induced using 0.1 mM IPTG and 0.2% arabinose in the presence of 1 mM pBPA (Bachem, F-2800.0001). After overnight expression, cells were harvested and lysed using the Bugbuster reagent (Novagen). Proteins were purified using Ni-NTA affinity chromatography followed by Strep-Tactin purification according to the manufacturer's instructions. Protein purity was confirmed by SDS-PAGE. Correct incorporation of pBPA was confirmed by Q-ToF LC-MS. Purified proteins were stored at −80° C. until use.

The pET28a plasmid encoding SUMO-TNFα-SB fusion protein was transformed into E. coli BL21 (DE3). Cells were cultured in LB medium (10 g peptone, 10 g NaCL, 5 g yeast extract per liter) containing 30 μg/mL kanamycin. Protein expression was induced using 0.1 mM IPTG. Cells were harvested after overnight expression and were resuspended in Binding Buffer (40 mM Tris-HCl, 125 mM NaCl, 5 mM imidazole, pH 8.0) containing Benzonase enzyme. Cell disruption was then performed by ultrasonication on ice with 5 pulses of 20 s set to an amplitude of 50%. Proteins were first purified using Ni-NTA affinity chromatography at 4° C. The His-SUMO-tag was cleaved from TNFα-SB by adding 2 pL of SUMO protease to the 1.5 mL elution fractions and dialyzing the solution overnight at 4° C. against SUMO protease reaction buffer (20 mM Tris-HCl, 150 mM NaCl, 1 mM DTT, pH 8.0) using a 5 kDa dialysis membrane. Cleaved TNFα-SB was obtained using Ni-NTA affinity chromatography to remove the cleaved His-SUMO-tag. Protein purity was confirmed by SDS-PAGE. Purified proteins were stored at −80° C. until use.

The NanoLuc-mNeonGreen fusion protein was prepared as described previously (Suzuki, K., et al., Five colour variants of bright luminescent protein for real-time multicolour bioimaging. Nature Communications, 2016. 7: p. 1-10). Proteins were purified using Ni-NTA affinity chromatography and Strep-Tactin purification. Purified proteins were stored at −80° C. until use.

### Photo-Conjugation

Anti-adalimumab/TNFα monoclonal antibody (ATantibody) was obtained from BioRad (HCA207). Photo-conjugation reactions were performed using a Promed UVL-30 UV light source (4×9 watt). Samples containing antibody and 2 equivalents of pG-LB in PBS buffer (pH 7.4) were illuminated with 365 nm light for 2 hours. The samples were kept on ice during photo-conjugation. The photo-conjugated products contained a mixture of unconjugated pG-LB and AT antibody conjugated with one or two copies of pG-LB protein and were used without further purification.

### Luminescent Assays

Adalimumab was ordered from Gentaur (A1048-100). Intensiornetric assays were performed at HCA207-pG-LB concentrations of 2.5-30 nM and TNFα-SB concentrations of 25-300 nM in a total volume of 15 μL in Greiner flat white 384-well plates. After incubation of sensor proteins and analytes for 0.5 hours, NanoGlo substrate (Promega, N1110) was added at a final dilution of 500-fold. Luminescence intensity was recorded on Tecan Spark 10M plate reader with an integration time of 100 ms. For ratiometric assays, 10-100 μM NanoLuc-mNeonGreen fusion protein was added into the samples and luminescence spectra were recorded between 398 nm and 653 nm with a step size of 15 nm, a bandwidth of 25 nm and an integration time of 100 ms. Experiments were done in buffer (PBS with 1 mg/ml BSA, pH 7.4) or in blood plasma diluted with PBS/BSA buffer.

### Sensor Design Synthesis of Sensor Components

In order to establish a proof-of-concept, adalimumab (ADL) was chosen as a target antibody. Adalimumab is currently the most widely used anti-TNFα antibody, and a representative of a family of TNFα blocking biopharmaceuticals widely used in treating inflammatory diseases that also include infliximab and its biosimilars, and hybrid proteins containing non-antibody receptor domains. The sensor according to this example has two parts; (1) a TNFα fusion protein in which the C-terminus of TNFα is linked via a flexible linker to a SB, and (2) a monoclonal antibody that binds the ADL-TNFα complex that is functionalized with a LB fragment using a photo-cross-linkable protein-linker-LB fusion protein previously reported. The high affinity of ADL for the native TNFα trimer (K_{d} =0.11 nM) ensures that all ADL will be form a complex with TNFα when an excess of the latter is used in the assay. The anti ADL-TNFα antibody used here was reported to bind the complex with a K_{d} of 67 nM (https://www.bio-rad-antibodies.com/anti-adalimumab-antibody-humira.html), which allowed the assay to be sensitive in the concentration range that is of interest for therapeutic drug monitoring (0.1-22 mg/L or 1-200 nM).

The DNA sequence of TNFα was taken from Hoffmann et al. (Recombinant production of bioactive human TNF-αby SUMO-fusion system-High yields from shake-flask culture. Protein Expression and Purification, 5 2010. 72(2): p. 238-243), who used an N-terminal His-SUMO-tag to obtain a high yield of the properly folded protein. This DNA sequence was cloned into a pET28a vector such that the C-terminus of TNFα is fused via a long, flexible glycine-serine linker to a SB variant with moderate affinity for the LB fragment (K_{d} of 2.5 μM). Attachment to the C-terminus of the protein should allow formation of the native trimeric complex without any steric hindrance of the fragment. Moreover, it was also important to provide sufficient steric freedom between both fragments such that the luciferase complementation could take place. The His-SUMO-TNFα-SB fusion protein was recombinantly expressed in E. coli and purified in good yield using nickel-affinity chromatography (see: FIG. 2). The His-SUMO tag was removed by overnight incubation of His-SUMO-TNFα-SB with SUMO protease, followed by a second nickel affinity chromatography step to separate TNFα-SB from His-SUMO and uncleaved HIS-SUMO-TNFα-SB protein. The flow through of this column contained essentially pure TNFα-SB fusion of the expected molecular weights (28 kDa). Conjugation of LB to the anti ADL-TNFα antibody was achieved by photo-crosslinking the antibody with 2 equivalents of pG-LB protein for 2 hours using 365 nm on ice. SDS-PAGE analysis revealed successful conjugation of most of the antibody with 1 or 2 copies of the pG-LB protein. The conjugation product was not further purified and used directly in the bioluminescent assay.

### Proof-of-Principle and Effect Of Sensor Concentration

To test the feasibility of the new sensor principle we performed an adalimumab titration experiment in buffer using 2.5 nM anti-AT-LB and 25 nM TNFα-SB. A large, 80-fold increase in luminescence intensity was observed between 0.01 and 10 nM ADL, after which the intensity remained constant. This impressive increase in luminescence demonstrates that the principle of the assay works and that the TNFα-SB proteins folded correctly and self-assembled into their native trimeric structure. Next the concentration of anti-AT-LB and TNFα-SB was systematically changed to find optimal assay conditions with respect to the increase in signal increase and the ADL sensitivity. FIGS. 3A-B show that increasing the anti-AT-LB concentration increases both the signal and the background luminescence signal, without significantly affecting the dynamic range. Increasing the concentration of TNFα-SB from 100 to 300 nM does not affect the assay performance.

### Measurements in Diluted Blood Plasma and Ratiometric Detection.

Ideally the assay should be able to measure ADL concentrations between 3 and 70 nM in blood plasma. Since the assay in buffer is most sensitive between 0.1 and 10 nM ADL, a protocol was established in which the plasma sample was diluted with buffer in a 1:3 ratio, corresponding to a 4-fold dilution. FIG. 4 shows the results of an ADL titration experiment in diluted blood plasma, where the concentrations of ADL on the X-axis represent the final concentrations after dilution.

The response curve and dynamic range are very similar to that obtained in pure buffer, showing that the assay is not affected by the presence of 25% (v/v) of blood plasma.

The data shown thus far represent intensiometric data in which the absolute intensity of the blue luminescent signal is measured as a signal for complex formation. However, the absolute signal intensity not only depends on the amount of ADL, but is also affected by environmental parameters such as pH, temperature and ionic strength. Most importantly, however, the signal depends on the concentration of furimazine substrate, which means that the signal will decrease in time as a result of substrate turn-over, most significantly at higher ADL concentration. This is particularly problematic for point-of-care applications where it may be difficult to carefully calibrate the assay for variations in sensor concentration, substrate turn-over and environmental conditions. Recently a new approach to convert intensiometric luciferase-based assays was reported such as these into ratiometric assays by simply adding an additional calibrator luciferase. The assay mixtures were spiked with a bioluminescent NanoLuc-mNeonGreen fusion protein which can use the same substrate furimazine and generate green light (Ni, Y. and M. Merkx, Ratiometric detection of luciferase assays using a calibrator luciferase. provisional patent application 62/864583, manuscript in preparation, 2020). In this way, the emission ratio as a function of ADL concentration negates the influence of the substrate concentration and the reaction conditions.

FIGS. 5A-B show an ADL titration experiment in 1:5 diluted blood plasma using 100 μM of NanoLuc-mNeonGreen as a calibrator luciferase in addition to 10 nM anti-AT-LB and 100 nM TNFα-SB. FIG. 5A shows that the blue peak of reconstituted split Nanoluc increases as function of ADL concentration, whereas the peak at 513 nm from the calibrator luciferase remains constant. The titration curve obtained by plotting the ratio of the blue and green emission peaks as function of ADL concentration shows a large response in the physiologically relevant concentration range, showing a 15-20 fold overall change in emission ratio and a LOD of approximately 25 μM (corresponding to 0.15 nM in plasma).

The ratiometric signal also allows one to reliably monitor the kinetics of complex formation, and thus establish the optimal incubation time. FIG. 6 shows the increase in emission ratio following addition of 0, 0.75, 3 or 15 nM ADL to a mixture containing all the sensor components and substrate. As expected, the kinetics of complex formation are concentration dependent, reaching equilibrium at approx. 10 min for the higher concentrations. The emission ratio in the absence of ADL is constant.

### Conclusion

Given the above, a generic homogenous immunoassay format based on the complementation of split Nanoluc luciferase was developed for direct detection of TNFα-binding antibodies. Proof of concept was provided by successful ratiometric detection of adalimumab in diluted blood plasma with a LOD of 25 μM and a large dynamic range. In contrast to the sandwich immunoassay format based on two antibodies, the assay format introduced here has a fusion protein of the target antigen with a SB fragment of NanoLuc luciferase and a single detection antibody, which is conjugated to the LB fragment of Nanoluc, that binds to the adalimumab-TNFα complex. This new assay format is particularly attractive for the detection of therapeutic antibodies that bind to large and/or structurally complex discontinuous epitopes. The assay reported here could be easily amended for the detection of other TNFα-binding antibodies by using a detection antibody that is specific for the target antibody-TNFα-complex. The sensor principle could be further extended to other antibodies, provided an antigen-SB fusion protein can be produced and a specific detection antibody is available that either specifically binds the target antibody (but not at the antigen binding site), or binds to the complex of the target antibody and its antigen.

### Example 3

### Development of Red-Shifted Calibrator Luciferase

To develop a red-shifted NanoLuc luciferase, a fluorophore-labelled NanoLuc construct with highly efficient bioluminescence resonance energy transfer (BRET) from NanoLuc to the fluorophore was developed. The Cy3 fluorophore has an emission maximum at 563 nm and its excitation spectrum has spectral overlap with NanoLuc emission, and therefore was chosen to be incorporated into the NanoLuc luciferase via maleimide-thiol coupling. To achieve this, the native cysteine (Cys175) in NanoLuc was first replaced by serine, followed by site-directed mutagenesis to introduce a cysteine residue either near the N-terminus, C-terminus or in the accessible loops where the protein folding and bioluminescent properties are supposed not to be disrupted. The BRET efficiency was then determined for each mutant after labelling with Cy3, indicating K9C, D157C and G191C as potent Cy3-conjugation sites. In order to further improve the BRET efficiency, double and triple-site mutants by combing these mutation positions were subsequently constructed for coupling multiple Cy3 to one NanoLuc protein. The triple mutant K9C/D157C/G191C was eventually obtained with decent Cy3 labelling yield of approximately 140% and high BRET efficiency. The K9C/D157C/G191C variant emits orange luminescence (FIG. 18A) captured by a digital camera and has an emission maximum at 568 nm (FIG. 18B).

The Cy3-labeled K9C/D157C/G191C was then used as a calibrator luciferase to carry out a ratiometric assay of cTnI by using 19C7-LB and 4C2-SB sensor proteins. This red-shifted calibrator enabled a maximal ratio change of 45, superior to the green calibrator NL-mNG, which gave a maximal ratio change of 25 in the ratiometric assay of cTnI (FIG. 19).

### DESCRIPTION OF THE DRAWINGS

FIG. 1A

Schematic representation of homogeneous bioluminescent sandwich immunoassay. In the presence of antigen, antibody conjugated sensor proteins bind to the antigen, driving the reconstitution of active NanoLuc.

FIG. 1B

Split-luciferase based detection of TNFα binding antibodies. The SB fragment of Nanoluc is fused via a semi-flexible linker to recombinantly expressed TNFα, while the LB fragment is fused to photo-cross-linkable protein G, allowing conjugation to a detection antibody that specifically binds to the adalimumab-TNFα complex.

FIG. 2

SDS-PAGE analysis of TNFα-SB purification and antibody-pG-LB photo-crosslinking.

FIGS. 3A-B

Intensiometric immunoassay for Adalimumab (FIG. 3A) Proof of principle using 2.5 nM anti-AT-LB and 25 nM TNFα-SB. Blue line indicates assay with all components, other colours indicate controls (FIG. 3B) adalimumab response curves using different concentrations of anti-AT-LB and TNFα-SB. All assays were done in PBS-BSA buffer using 500-fold diluted furimazine. Error bars represent mean±SD (n=3).

FIG. 4

Intensiometric immunoassay for adalimumab in 1:3 diluted plasma using 10 nM anti-AT-LB and 100 nM TNFα-SB. Blue line indicates assay with all components, other colours indicate controls. All assays were done in PBS-BSA buffer mixed with blood plasma at a 1:3 ratio using 500-fold diluted furimazine. Error bars represent mean±SD (n=3).

FIGS. 5A-B

Ratiometric immunoassay for Adalimumab in 1:5 diluted plasma using 10 nM anti-AT-LB, 100 nM TNFα-SB and 100 μM NanoLuc-mNeonGreen. Emission spectra at different ADL concentrations (FIG. 5A). Emission ratio plotted as a function of ADL concentration (FIG. 5B). Blue line indicates assay with all components, other colors indicate controls. All assays were done in PBS-BSA buffer mixed with blood plasma at a 1:5 ratio using 500-fold diluted furimazine. Error bars represent mean±SD (n=3).

FIG. 6

Ratiometric assay to monitor the kinetics of complex formation at various ADL concentrations. All assays were done using 10 nM anti-AT-LB, 100 nM TNFα-SB and 100 μM NanoLuc-mNeonGreen in PBS-BSA buffer mixed with blood plasma at a 1:5 ratio using 500-fold diluted furimazine.

FIG. 7

DNA and amino acid sequence of pG-LB. Strep-tag (gray), protein G (red), amber stop codon (yellow), LB (cyan), His-tag (dark red).

FIG. 8

DNA and amino acid sequence of His-SUMO-TNFα-SB. His-tag (dark red), SUMO-tag (yellow), TNFα (pink), SB (cyan), Strep-tag (gray).

FIG. 9

DNA and amino acid sequence of pG-SB. Strep-tag (gray), protein G (red), amber stop coden (yellow), SB (blue), His-tag (dark red).

FIG. 10

DNA and amino acid sequences of SB in pG-SB variants.

FIG. 11

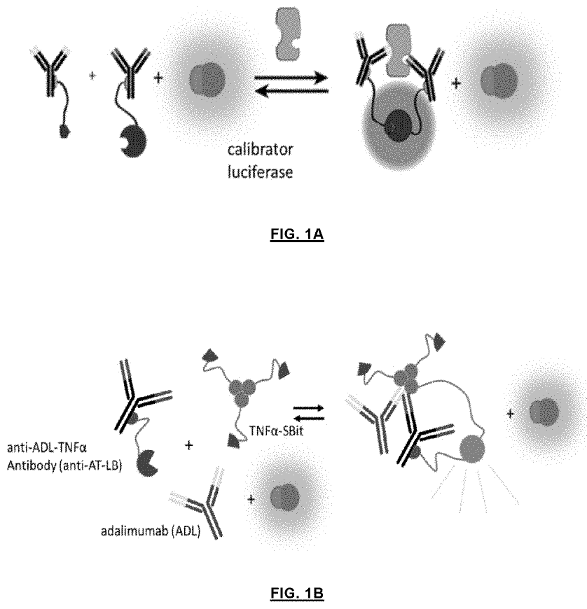

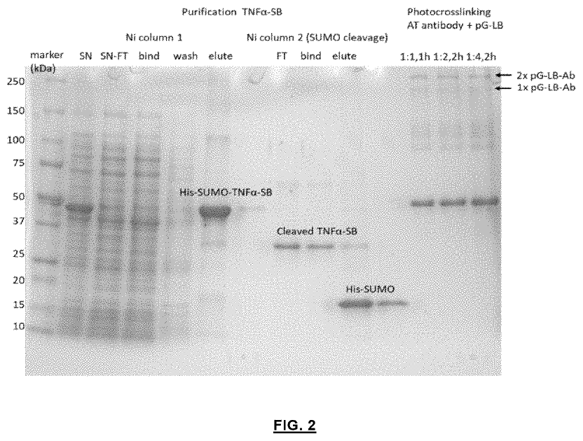

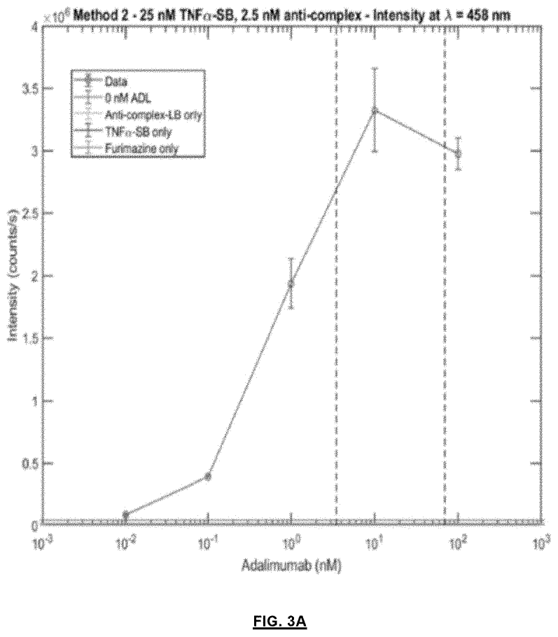

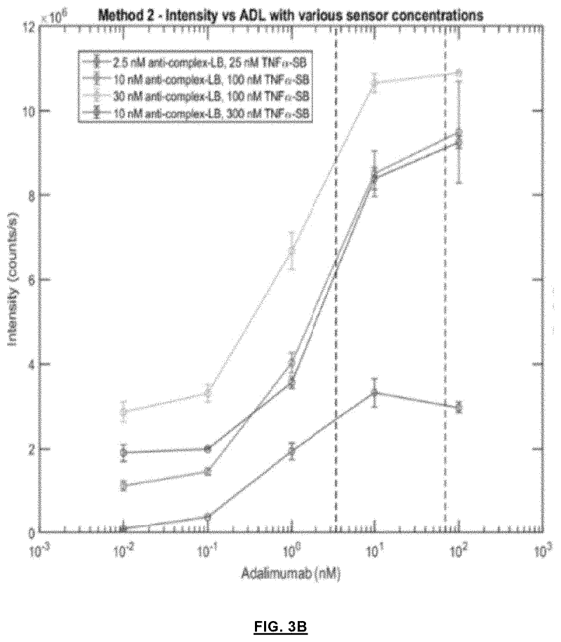

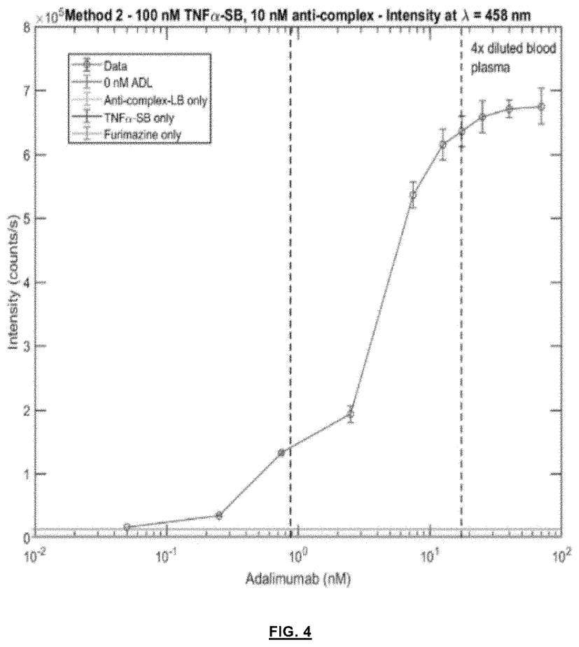

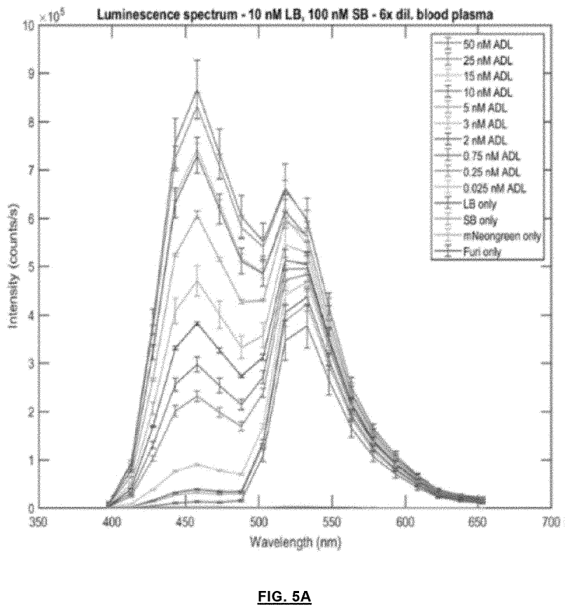

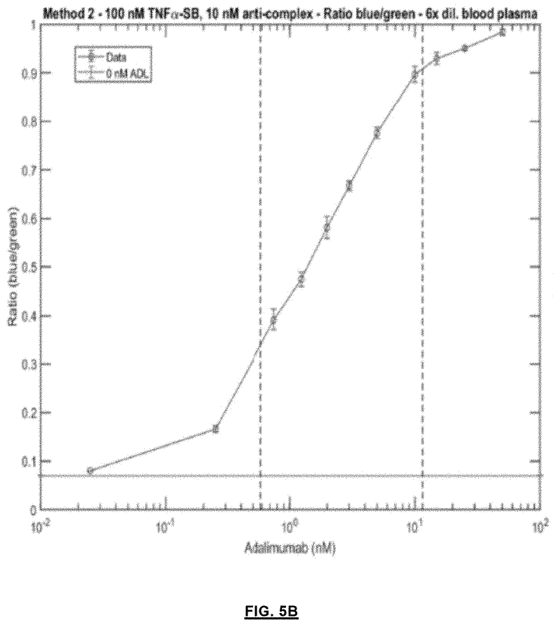

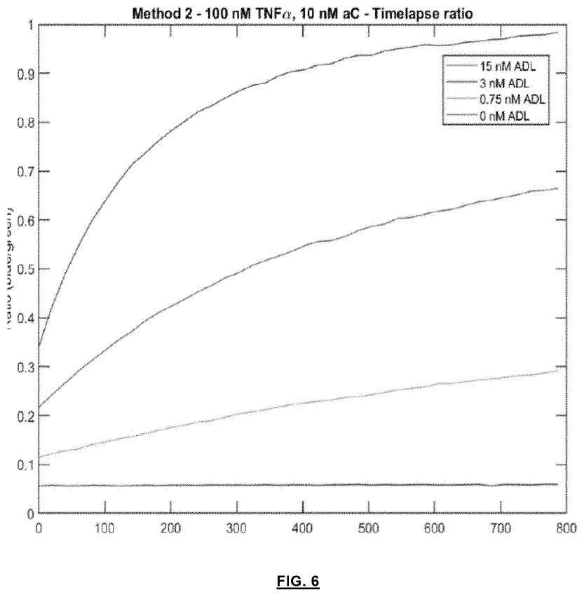

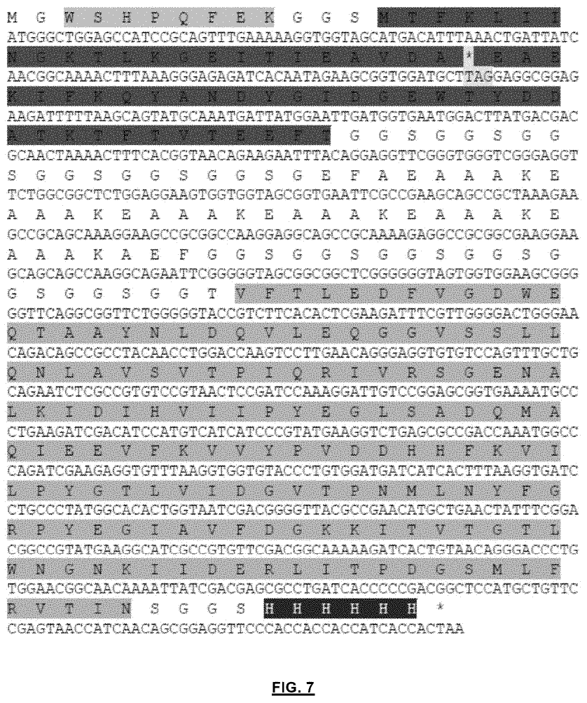

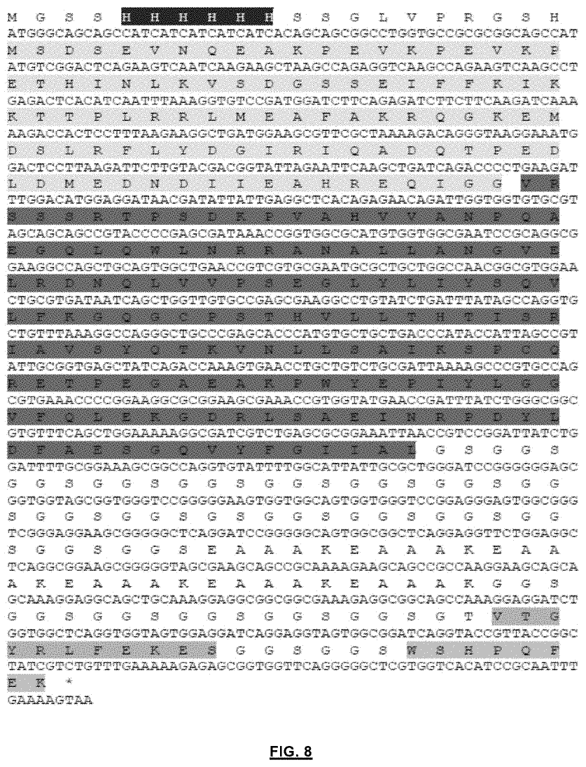

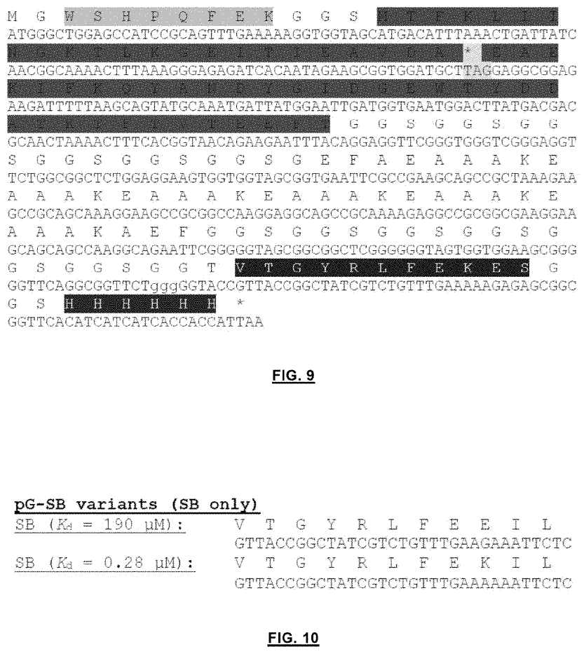

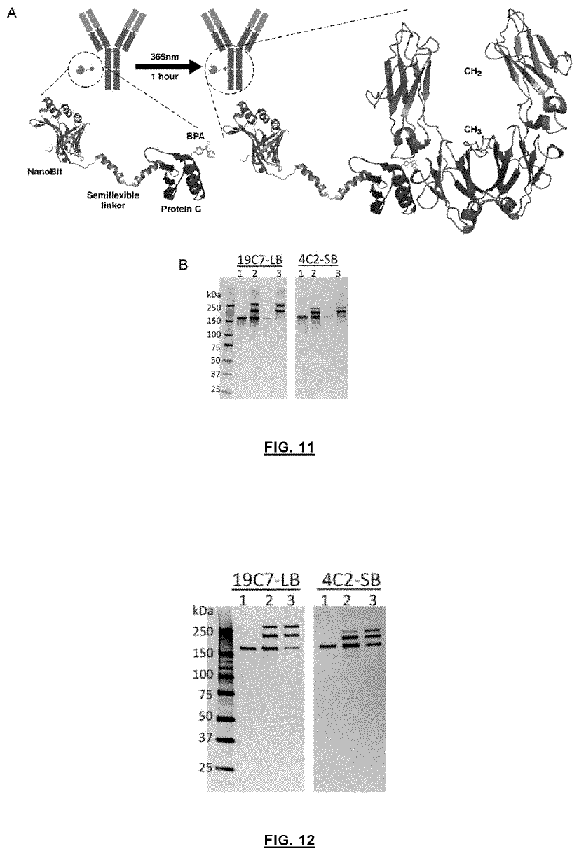

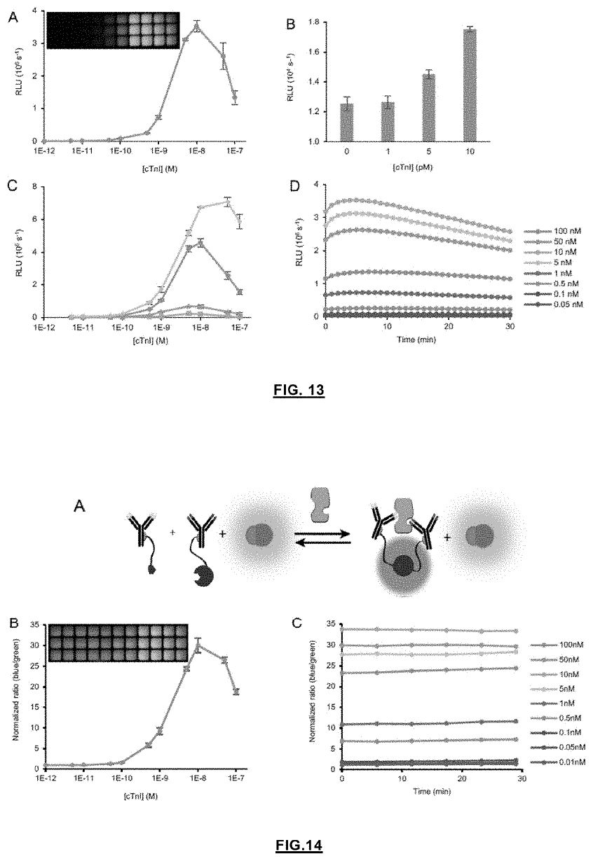

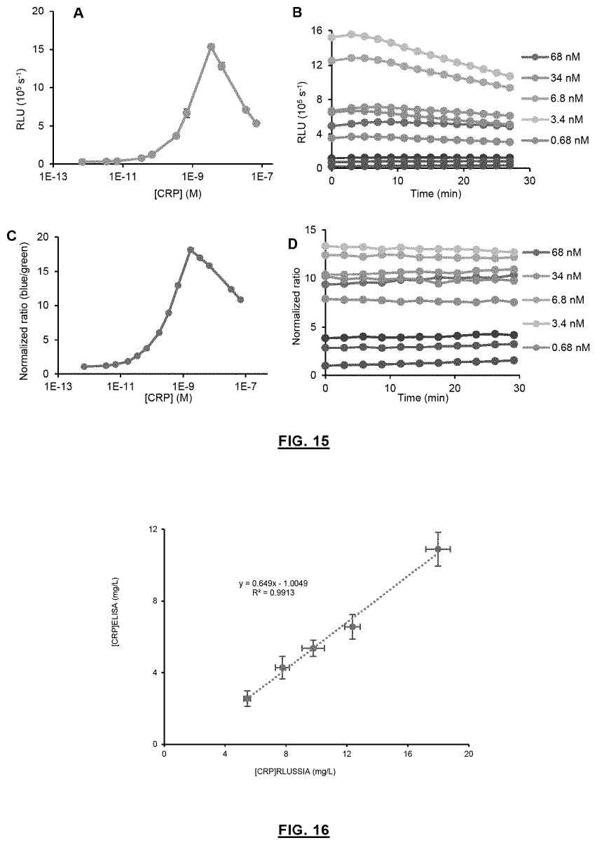

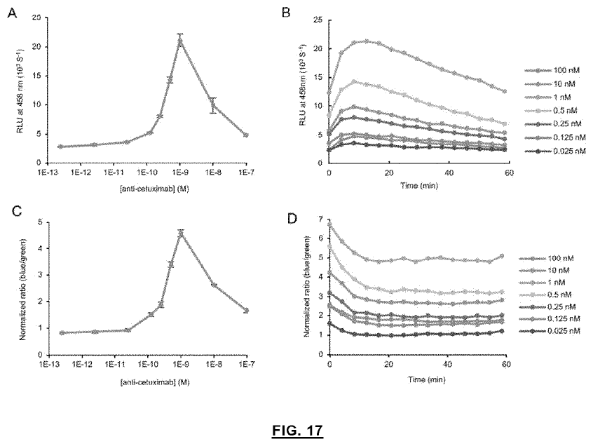

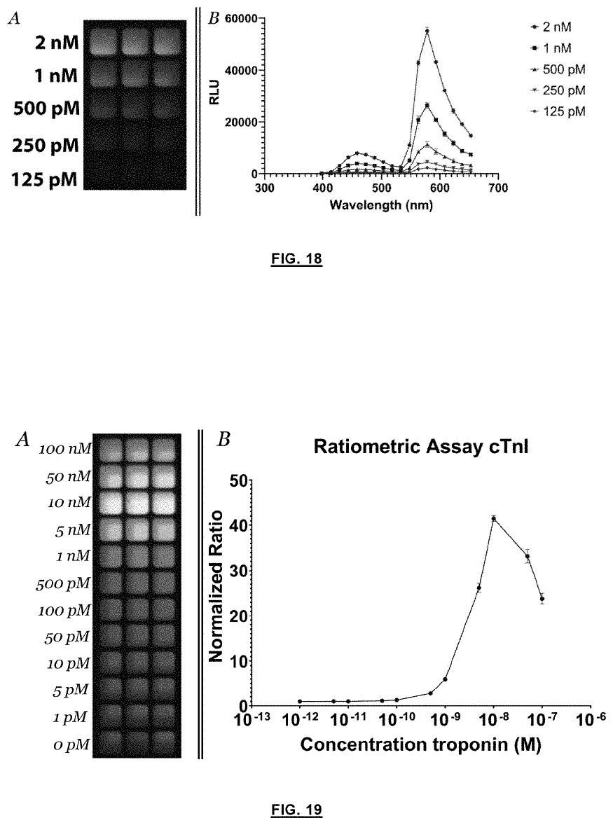

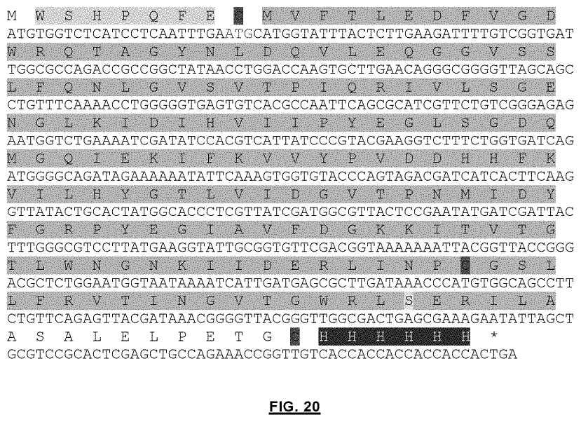

Photo-conjugation of sensor proteins. Protein G-mediated site specific conjugation by UV illumination (FIG. 11A). Protein G contains the unnatural amino acid BPA (in red) and is fused to split NanoLuc (LB or SB) via a semiflexible linker. When bound to the Fc domain of antibody and activated by UV light, the protein G fusion protein is covalently conjugated to the antibody. Either one or two protein G adapters can be photo-crosslinked to the Fc domain. SDS-PAGE analysis of the purified photo-conjugated products (FIG. 11B). Lane 1, anti-cTnI 19C7 or 4C2; Lane 2, photo-conjugated products; Lane 3, purified photo-conjugated proteins.

FIG. 12

SDS-PAGE analysis of the photo-conjugated products by using different molar ratios of pGLB/SB to antibody. Lane 1, antibody; Lane 2, photo-conjugated products using equal molar ratio of antibody to pG-LB/SB; Lane 3, photo-conjugated products using 1:2 molar ratio of antibody to pG-LB/SB.

FIG. 13

Intensiometric immunoassay of cTnI. FIG. 13A: cTnI dose-dependency using 1 nM 19C7-LB and 4C2-SB. Inset: picture of the samples. FIG. 13B: limit of detection. FIG. 13C: cTnI dose-dependency using different sensor concentrations. (▪) 0.1 nM 19C7-LB and 0.1 nM 4C2-SB; (▴) 0.1 nM 19C7-LB and 1 nM 4C2-SB; (●)1 nM 19C7-LB and 1 nM 4C2-SB; (♦) 1 nM 19C7-LB and 10 nM 4C2-SB. FIG. 13D: time course of luminescence signal in the presence of different concentrations of cTnI. Error bars represent mean±SD (n=3).

FIG. 14

FIG. 14A: schematic representation of the ratiometric assays with NanoLuc-mNeonGreen fusion protein as a calibrator luciferase. FIG. 14B: cTnI dose-dependency using 1 nM 19C7-LB and 4C2-SB spiked with 5 μM NanoLuc-mNeonGreen fusion protein. Inset: picture of the samples. FIG. 14C: time course of emission ratio in the presence of different concentrations of cTnI.

FIG. 15

Bioluminescent immunoassay of CRP. FIG. 15A: CRP dose-dependency using 1 nM C6-LB and 10 nM C135-SB. FIG. 15B: time course of luminescence signal in the presence of different concentrations of CRP. FIG. 15C: CRP dose-dependency using 1 nM C6-LB and 10 nM C135-SB spiked with 2 μM NanoLuc-mNeonGreen fusion protein in buffer. FIG. 15D: time course of emission ratio in the presence of different concentrations of CRP.

FIG. 16

Correlation of CRP concentrations measured by ELISA and assay of the inventors. The samples were prepared in blood plasma, diluted 1/50 in buffer and quantified using calibration curves obtained in buffer. Error bars represent mean±SD (n=4).

FIG. 17

Bioluminescent immunoassays of anti-cetuximab. FIG. 17A: intensiometric assays using 1 nM cetuximab-LB and cetuximab-SB. FIG. 17B: kinetics of intensiometric assays with different concentrations of anti-cetuximab. FIG. 17C: ratiometric assays using 1 nM cetuximab-LB and cetuximab-SB spiked with 1 μM NanoLuc-mNeonGreen fusion protein. FIG. 17D: kinetics of ratiometric assays with different concentrations of anti-cetuximab.

FIG. 18

Luminescence of the red-shifted NanoLuc at different concentrations. FIG. 18A: picture of taken by a digital camera; FIG. 18B: luminescence spectra measured by a plate reader.

FIG. 19

Ratiometric assay of cardiac troponin I using 1 nM 19C7-LB and 4C2-SB spiked with 1 nM red-shifted NanoLuc. FIG. 19A: picture of the samples taken by a digital camera; FIG. 19B: normalized blue/red ratio measured by plate reader.

FIG. 20

DNA and amino acid sequence of K9C/D157C/G191C variant. Strep-tag (gray), NanoLuc (cyan), cysteine (red), His-tag (dark red).

1-15. (canceled)

16. A bioluminescent assay for the ratiometric quantification of an analyte of interest, comprising a detector luciferase that is reactive to a substrate to emit light at a first wavelength and wherein the detector luciferase is responsive to the analyte of interest, wherein the bioluminescent assay further comprises a calibrator luciferase, wherein the calibrator luciferase is reactive to the same substrate to emit light at a second wavelength which second wavelength is different from the first wavelength emitted by the detector luciferase.

17. The bioluminescent assay according to claim 16, wherein the calibrator luciferase is functionalized with a fluorescent acceptor to allow energy transfer from the luciferase domain of the calibrator luciferase to the fluorescent acceptor.

18. The bioluminescent assay according to claim 16, wherein the calibrator luciferase is non-responsive to the analyte of interest.

19. The bioluminescent assay according to claim 16, wherein the calibrator luciferase is a variant of the detector luciferase and contains the same luciferase domain as the detector luciferase.

20. The bioluminescent assay according to claim 16, wherein the bioluminescent assay is selected from the group consisting of bioluminescent sandwich immunoassay, competitive immunoassays, sensor proteins based on allosteric modulation of luciferase activity or reversible control a luciferase inhibition, transcription regulation assays, assays screening for protein-protein interactions, and DNA or RNA detection assays.

21. The bioluminescent assay according to claim 16, wherein the luciferase domain of the detector luciferase and calibrator luciferase is selected from the group consisting of luciferase domains of NanoLuc luciferase, Firefly luciferase, Renilla luciferase, Gaussia luciferase, TurboLuc luciferase and Aluc luciferase.

22. The bioluminescent assay according to claim 16, wherein the fluorescent acceptor is selected from the group consisting of an mNeonGreen protein, a fluorescent protein, a Cy3, a fluorescent dye, a fluorescent quantum dot, a fluorescent nanoparticle, or a carbon dot.

23. The bioluminescent assay according to claim 16, wherein the analyte of interest is selected from the group consisting of an antigen, an antibody, and a ligand.

24. The bioluminescent assay according to claim 16, usable in a method for quantifying an analyte of interest in a sample.

25. The bioluminescent assay according to claim 16, the calibrator luciferase in the bioluminescent assay usable in a method for quantifying an analyte of interest in a sample.

26. A method for quantifying an analyte of interest in a sample, comprising the steps of:
i) providing a sample comprising the analyte of interest;
ii) providing a detector luciferase and a calibrator luciferase, wherein:
the detector luciferase is reactive to a substrate to emit light at a first wavelength and wherein the detector luciferase is responsive to the analyte of interest; and
the calibrator luciferase is reactive to the same substrate to emit light at a second wavelength which second wavelength is different from the first wavelength emitted by the detector luciferase;
iii) measuring the bioluminescence intensity of the detector luciferase and the bioluminescence intensity of the calibrator luciferase for the sample provided in step i);
iv) calibrating the ratio of the measured bioluminescence intensity of the detector luciferase and the bioluminescence intensity of the calibrator luciferase; and
v) quantifying the analyte of interest based on the ratio calibrated in step iv).

27. The method according to claim 26, wherein the calibrator luciferase is functionalized with a fluorescent acceptor to allow energy transfer from the luciferase domain of the calibrator luciferase to the fluorescent acceptor.

28. The method according to claim 26, wherein the calibrator luciferase is non-responsive to the analyte of interest.

29. The method according to claim 26, wherein the calibrator luciferase is a variant of the detector luciferase and contains the same luciferase domain as the detector luciferase.

30. The method according to claim 26, wherein:
the luciferase domain of the detector luciferase and calibrator luciferase is selected from the group consisting of luciferase domains of NanoLuc luciferase, Firefly luciferase, Renilla luciferase, Gaussia luciferase, TurboLuc luciferase and Aluc luciferase; and/or
the fluorescent acceptor is selected from the group consisting of an mNeonGreen protein, a fluorescent protein, a Cy3, a fluorescent dye, a fluorescent quantum dot, a fluorescent nanoparticle, or a carbon dot.
